(19) United States
(12) Patent Application Publication
(10) Pub. No.: US 2024/0207572 A1
(43) Pub. Date: Jun. 27, 2024

(54) CATHETER TIP WITH HIGH BOND STRENGTH

(71) Applicants: Boston Scientific Medical Device Limited, Galway (IE); Boston Scientific Scimed, Inc., Maple Grove, MN (US)

(72) Inventors: Anshul Chabra, Delhi (IN); Robert M. Wold, Maple Lake, MN (US); Jahnavi Konduru, New Delhi (IN); Gagan Kumar, Gurgaon (IN)

(73) Assignees: Boston Scientific Medical Device Limited, Galway (IE); Boston Scientific Scimed, Inc., Maple Grove, MN (US)

(21) Appl. No.: 18/392,329

(22) Filed: Dec. 21, 2023

Related U.S. Application Data
(60) Provisional application No. 63/435,057, filed on Dec. 23, 2022.

(51) Int. Cl. A61M 25/00

(52) U.S. Cl. CPC A61M 25/0068; A61M 25/001; A61M 25/0012; A61M 25/0013; A61M 25/0052

(57) ABSTRACT

A catheter includes an elongated tubular shaft having a distal end, a proximal end, and a lumen extending therethrough. The elongated tubular shaft includes a polymeric outer layer, a polymeric inner layer, and a braided middle layer. A marker band is positioned adjacent the distal end of the elongated tubular shaft and may surround the middle layer, and a polymeric distal tip is attached to the distal end of the elongated tubular shaft. A distal end of the outer layer terminates proximal to the marker band such that the middle layer includes an exposed region that is exposed from the outer layer between the distal end of the outer layer and a proximal end of the marker band, and the distal tip is formed over the exposed region of the middle layer and the marker band, and extends distally beyond the distal end of the elongated tubular shaft.

### CROSS-REFERENCE TO RELATED APPLICATIONS

This application claims the benefit of U.S. Provisional Patent Application Ser. No. 63/435,057, filed Dec. 23, 2022, which is incorporated herein by reference.

### TECHNICAL FIELD

The present disclosure pertains to catheters for delivery of therapeutic agents or devices to a site within a body lumen, and associated accessories. More particularly, the present disclosure pertains to catheter tips with a high bond strength.

### BACKGROUND

A wide variety of intravascular catheters are known, including small diameter catheters having a central lumen therethrough that are configured for use in smaller vasculature. Such catheters are known as microcatheters. Catheters and microcatheters typically include an atraumatic distal tip that is free from a metal reinforcement. The distal tip is separately bonded to a distal end of a catheter shaft, thereby forming a butt joint. The problem with a butt joint is the bond between the distal tip and the catheter shaft is weak, inconsistent, and has a low extension at break. A need remains for improved catheter distal tips and bonding methods.

There is an ongoing need to provide alternative medical devices as well as alternative methods for manufacturing and using medical devices.

### BRIEF SUMMARY

This disclosure provides design, material, manufacturing method, and use alternatives for medical devices. An example medical device may include a catheter. The catheter may include an elongated tubular shaft having a distal end, a proximal end, and a lumen extending therethrough. The elongated tubular shaft may include a polymeric outer layer, a polymeric inner layer, and a braided middle layer positioned between the inner layer and the outer layer. A marker band may be positioned adjacent the distal end of the elongated tubular shaft and may surround the middle layer. A polymeric distal tip may be attached to the distal end of the elongated tubular shaft. A distal end of the outer layer may terminate proximal to the marker band such that the middle layer includes an exposed region that is exposed from the outer layer between the distal end of the outer layer and a proximal end of the marker band, and the distal tip may be formed over the exposed region of the middle layer and the marker band, and may extend distally beyond the distal end of the elongated tubular shaft.

Alternatively or additionally to any of the embodiments above, the outer layer may terminate at a distance in the range of about 0.5 mm (millimeters) to about 1.5 mm proximal of the marker band.

Alternatively or additionally to any of the embodiments above, the distal tip may provide a tensile strength in the range of 1.42 N (Newtons) to 1.55 N.

Alternatively or additionally to any of the embodiments above, the distal tip may provide a tensile strength of about 1.5 N.

Alternatively or additionally to any of the embodiments above, the distal tip may be formed from a polyether-block-amide copolymer.

Alternatively or additionally to any of the embodiments above, a proximal portion of the distal tip may surround and overlap with a distal portion of the outer layer.

Alternatively or additionally to any of the embodiments above, the distal tip may be formed from a coextrusion comprising a first polyether-block-amide copolymer layer and a second polyether-block-amide copolymer layer surrounding the first polyether-block-amide copolymer layer.

Alternatively or additionally to any of the embodiments above, a ratio of a radial thickness of the first polyether-block-amide copolymer layer to the second polyether-block-amide copolymer layer may be about 1:1.

Alternatively or additionally to any of the embodiments above, a ratio of a radial thickness of the first polyether-block-amide copolymer layer to the second polyether-block-amide copolymer layer may be about 1:1.4.

A method of forming a catheter having a distal tip may include disposing a braided middle layer over a polymeric inner layer, positioning a marker band over the braided middle layer, and extruding a polymeric outer layer over a proximal region of the braided middle layer while leaving a distal region of the braided middle layer exposed from the outer layer. The exposed distal region of the braided middle layer may extend between a distal end of the outer layer and a proximal end of the marker band. The method may further include cutting away portions of the braided middle layer and the inner layer extending distal of a distal end of the marker band. Thereafter, the method may include extruding a distal tip over the exposed distal region of the braided middle layer and the marker band. The distal tip may extend distally beyond the distal end of the marker band.

Alternatively or additionally to any of the embodiments above, the distal tip may be formed from a coextrusion comprising a first polyether-block-amide copolymer layer and a second polyether-block-amide copolymer layer surrounding the first polyether-block-amide copolymer layer.

Alternatively or additionally to any of the embodiments above, the distal tip may be bonded to the inner layer between a plurality of interstices in the exposed distal region of the middle layer.

Alternatively or additionally to any of the embodiments above, the distal tip may provide a tensile strength in the range of 1.42 N (Newtons) to 1.55 N.

Alternatively or additionally to any of the embodiments above, the outer layer may terminate at a distance in the range of about 0.5 mm (millimeters) to about 1.5 mm proximal of the marker band.

Alternatively or additionally to any of the embodiments above, an outer diameter of a distal end region of the outer layer may taper distally.

Alternatively or additionally to any of the embodiments above, a proximal portion of the distal tip may surround and overlap with the distal end region of the outer layer.

In another example, a catheter may include an elongated tubular shaft having a distal end, a proximal end, and a lumen extending therethrough. The elongated tubular shaft includes a polymeric inner layer having an inner surface defining the lumen of the elongated tubular shaft. The inner layer extends continuously from the proximal end of the elongated tubular shaft to the distal end of the tubular shaft. A braided middle layer surrounds the inner layer. The middle layer extends continuously from the proximal end of the elongated tubular shaft to the distal end of the elongated tubular shaft. A polymeric outer layer surrounds the middle layer. The outer layer extends continuously from the proximal end of the elongated tubular shaft to a distal end of the outer layer that is located proximal of the distal end of the elongated tubular shaft such that the middle layer includes a distal end region extending distal of the distal end of the outer layer. A polymeric distal tip may be extruded over the distal end region of the middle layer and extends distally beyond the distal end of the elongated tubular shaft.

Alternatively or additionally to any of the embodiments above, a proximal portion of the distal tip may surround and overlap with a distal portion of the outer layer.

Alternatively or additionally to any of the embodiments above, an outer diameter of the distal portion of the outer layer may taper distally.

Alternatively or additionally to any of the embodiments above, a marker band may be positioned adjacent the distal end of the elongated tubular shaft and surrounding the middle layer, wherein the distal tip may extend over and surround the marker band.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a plan view of a catheter in accordance with an embodiment of the disclosure;

FIG. 2 is a side view of a distal end of a catheter;

FIG. 2A is a longitudinal cross-sectional view of the distal end of the catheter of FIG. 2, taken along line 2A-2A;

FIG. 2B is an enlarged view of an exposed region, as in Circle 2B in FIG. 2A;

FIG. 3 is a transverse cross-sectional view of an exemplary distal tip of a catheter;

FIG. 3A is an enlarged cross-section of the wall of the distal tip of the catheter of FIG. 3;

FIG. 4 is a transverse cross-sectional view of an exemplary distal tip of a catheter;

FIG. 4A is an enlarged cross-section of the distal tip of the catheter of FIG. 4;

FIGS. 5A to 5D depict an illustrative method of forming a catheter having a distal tip;

FIG. 6 is an illustrative graph depicting a distal outer diameter profile of a catheter formed in accordance with this disclosure;

FIG. 7 is an illustrative graph comparing tensile force of a catheter with a butt joint versus a lap joint formed in accordance with this disclosure; and

FIG. 8 is an illustrative graph comparing an extension at break for a catheter with a butt joint versus a lap joint formed in accordance with this disclosure.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit of the disclosure.

### DETAILED DESCRIPTION

For the following defined terms, these definitions shall be applied, unless a different definition is given in the claims or elsewhere in this specification. All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes, 1, 1.5, 2, 2.75, 3, 3.80, 4, and 5). As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in this specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the claims.

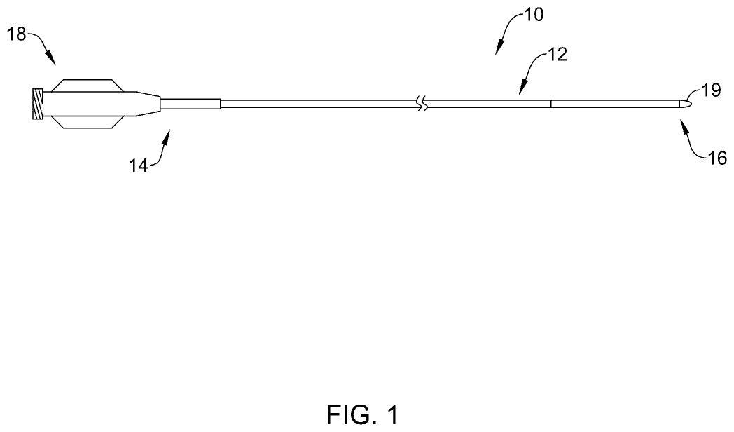

Catheters, such as microcatheters, may be used to access various regions of the vasculature and other body lumens. For example, some catheters are configured for a variety of therapeutic treatments such as diagnosis of vascular complications, delivery of an embolic treatment, delivery of a medical device, and intravascular mapping. FIG. 1 is an example of such a catheter 10. As shown in FIG. 1, the catheter 10 may include an elongated tubular shaft 12 having a distal end 16 and a proximal end 14. The catheter 10 may include a hub 18 secured to the proximal end 14 of the elongated tubular shaft 12. The catheter shaft 12 of the catheter 10 may have any desired length and outer diameter. For example, the catheter shaft 12 may have a length that is in the range of about 50 to about 200 centimeters and may have an outer diameter (OD) that is less than 3 French, for example. In some cases, the catheter shaft 12 may have an OD of 1.7 French or less, 2 French or less, 2.5 French or less, 2.8 French or less, or any other suitable outer diameter. In some cases, the catheter shaft 12 may have an OD greater than 3 French. For example, the catheter shaft 12 may have an OD in the range of about 3 French to 10 French or more. In some cases, the catheter shaft 12 may include an OD of about 2.6 French along a distal end region, and an OD of about 2.8 French along a proximal end region. In some cases, the catheter shaft 12 may have an inner diameter (ID) sized to accommodate a 0.014-inch guidewire, a 0.018-inch guidewire, or a 0.035-inch guidewire, for example. For instance, the ID may be about 0.015 inches to about 0.017 inches, about 0.019 inches to about 0.022 inches, or about 0.034 inches to about 0.038 inches, for example. In other cases, the catheter 10 may have an ID of about 0.015 inches, about 0.019 inches, about 0.036 inches, or any other suitable inner diameter. These sizes may vary depending on particular usage.

In some cases, a distal tip 19 may be attached to and extend distal of the distal end 16 of the elongated tubular shaft 12. The distal tip 19 may be a polymeric distal tip 19, which may be formed from an elastomer (e.g., Pebax®), a thermoplastic polymer, or any other suitable polymer. The distal tip 19 may be formed of a softer material (i.e., lower durometer) than other portions of the elongate shaft 12 of the catheter 10, such as by using a polymer or elastomer having a shore hardness of less than 40D, for example. In some cases, the distal tip 19 may be formed from a polymer with a shore hardness of about 35D or less. In some instances, the distal tip 19 may have a length of about 1 millimeter (mm) to about 2 mm, or about 1 mm to about 5 mm, for example. In some cases, the distal tip 19 may have a length of about 2.0 mm, about 1.7 mm, about 1.5 mm, about 1.3 mm, about 1.0 mm, or any other suitable length. The distal tip 19 may include an outer diameter that is sized to match the OD of the catheter shaft 12 proximal of the distal tip 19. In some cases, the distal tip 19 may include an OD of 1.7 French or less, 2 French or less, 2.5 French or less, 2.8 French or less, or any other suitable diameter. In some examples, the elongate shaft 12 may transition in hardness along the length of the elongate shaft 12. For instance, the elongate shaft 12 may include a plurality of segments having varying hardness, such as using Pebax® in varying hardnesses (for example, 75D to 63D to 55D to 45D from proximal to distal along the elongate shaft 12, to the distal tip). These are just examples.

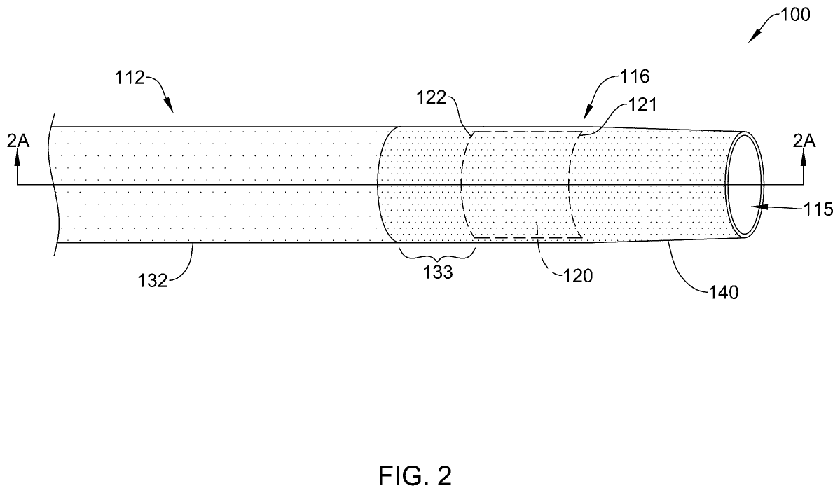

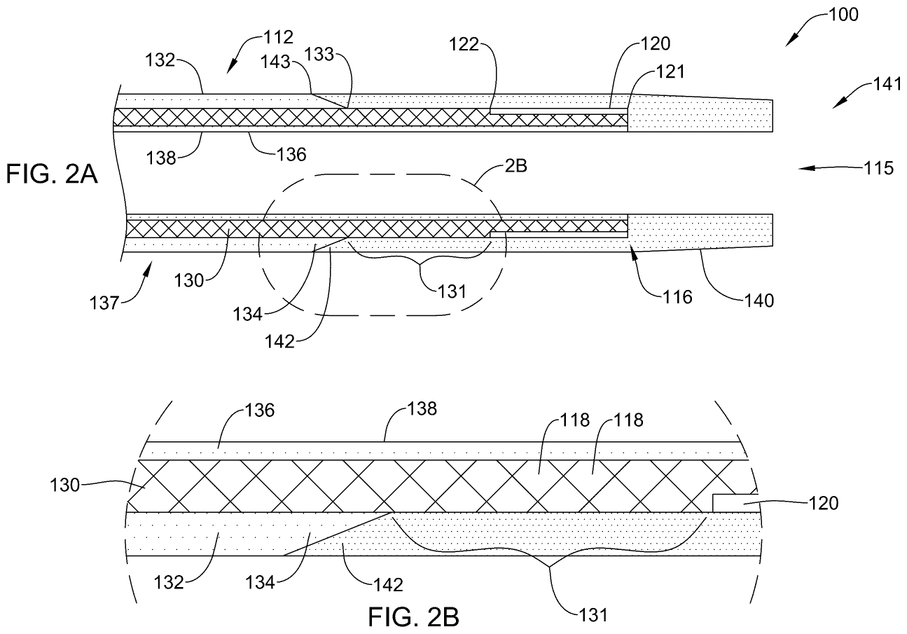

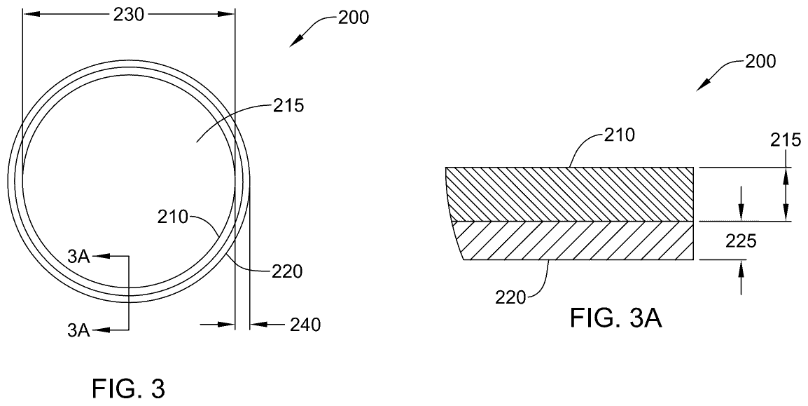

FIG. 2 is a side view of a distal end region of an elongated tubular shaft 112 of a catheter 100, FIG. 2A is a longitudinal cross-sectional view of the distal end region of the elongated tubular shaft 112 of the catheter 100 of FIG. 2, taken along line 2A-2A, and FIG. 2B is an enlarged view of a portion of the elongate shaft 112 taken from FIG. 2A. The elongated tubular shaft 112 may be an example of the elongated tubular shaft 12 shown in FIG. 1. The elongated tubular shaft 112 may include an outer layer 132, an inner layer 136, and a middle layer 130 positioned between the outer layer 132 and the inner layer 136. The elongated tubular shaft 112 may include a lumen 115 extending therethrough. The lumen 115 may be considered as a guidewire lumen, as well as an injection lumen, or the like. As an example, in use, a practitioner may insert the catheter (e.g., catheter 10) over a guidewire (not shown). Once the target vessel is reached, the guidewire may be removed and a fluid may be injected at the target site through the lumen 115. In some cases, the inner layer 136 may include an inner surface 138 defining the lumen 115 of the elongated tubular shaft 112.

The inner layer 136 of the elongated tubular shaft 112 may be a polymeric inner layer, and may be formed from or include a coating of a material having a suitable low coefficient of friction. Examples of suitable materials may include a polymer such as polytetrafluoroethylene (PTFE). The inner layer 136 may be dimensioned to define the lumen 115, having an appropriate inner diameter to accommodate its intended use. In some cases, the inner layer 136 may define the lumen 115 having a diameter of about 0.015 inches to about 0.040 inches, for example. The inner layer 136 may extend continuously from a proximal end (e.g., proximal end 14) of the elongated tubular shaft 112 to the distal end 116 of the elongated tubular shaft 112.

The outer layer 132 of the elongated tubular shaft 112 may be a polymeric outer layer, and may be formed from a polymer that may provide desired flexibility and strength. In some cases, the outer layer 132 may be formed from a nylon polymer, a thermoplastic polymer, elastomeric polyamides, or any other suitable polymer. The outer layer 132 may be dimensioned to define the outer diameter of the elongated tubular shaft 112. In some cases, the outer diameter of the elongated tubular shaft 112 may be less than 3 French, for example. In some cases, the outer layer 132 may have an OD of 1.7 French or less, 2 French or less, 2.5 French or less, 2.8 French or less, or any other suitable outer diameter. The outer layer 132 may surround the middle layer 130, and may extend continuously from a proximal end (e.g., proximal end 14) of the elongated tubular shaft 112 to a distal end 133 of the outer layer 132 that is located proximal of the distal end 116 of the elongated tubular shaft 112, such that the middle layer 130 includes an exposed distal end region 131 (generally referred to herein as exposed region 131), extending distal of the distal end 133 of the outer layer 132.

The middle layer 130 may be positioned radially between the outer layer 132 and the inner layer 136, and may be formed of a reinforcing structure, such as a braid or coil. The middle layer 130 may be considered to be a reinforcing layer that increases the torque response of the elongated tubular shaft 112. The middle layer 130, or filaments thereof, may be formed of any suitable material, such as stainless steel, tungsten, gold, titanium, silver, copper, platinum, or nitinol. In some cases, the middle layer 130 may be formed from a non-metallic material such as polymer fibers, glass fibers, or liquid crystal polymer (LCP) fibers. The middle layer 130, when provide as a braided reinforcement layer, may be formed using a variety of different weave patterns, such as a one-over-one-under pattern, a two-over-two-under pattern, a three-over-three-under pattern, a four-over-four-under pattern, or the like. In some cases, the middle layer 130 may be formed using a two-over-two-under configuration in which each filament extends over two intersecting filaments, then extends under the next two intersecting filaments, then extends over the next two intersecting filaments, etc. The middle layer 130 may surround the inner layer 136, and may extend continuously from a proximal end (e.g., proximal end 14) of the elongated tubular shaft 112 to the distal end 116 of the elongated tubular shaft 112.

In some cases, a marker band 120 may be positioned adjacent a distal end 116 of the elongated tubular shaft 112, and may surround the middle layer 130, as shown in FIG. 2A. The distal end of the marker band 120 may be substantially aligned with the distal end 116 of the elongated tubular shaft 112. In some cases, the marker band 120 may be formed from a radiopaque material, such as gold, tungsten, a tungsten filled polymer, or the like so as to be visible under fluoroscopy. In some cases, an outer diameter of the outer layer 132 may taper distally at the distal end 133 of the outer layer 132. In other words, a distal end region of the outer layer 132 may taper radially inward in a distal direction to the distal end 133 of the outer layer 132. Thus, the outer layer 132 may terminate proximal to the marker band 120 such that the distal end 133 of the outer layer 132 may terminate at a distance proximal of the marker band 120. For example, the outer layer 132 may terminate at a distance in the range of about 0.5 mm (millimeters) to about 1.5 mm, or in the range of about 0.5 mm to about 3 mm proximal of the marker band 120 in some instances. In some cases, when the outer layer 132 terminates proximal of the marker band 120, the middle layer 130 may include the exposed region 131 that is exposed from the outer layer 132 between the distal end 133 of the outer layer 132 and a proximal end 122 of the marker band 120. In other words, the middle layer 130 extends distally beyond the distal end 133 of the outer layer such that the outer layer 132 does not surround or extend over the exposed region 131 of the middle layer 130.

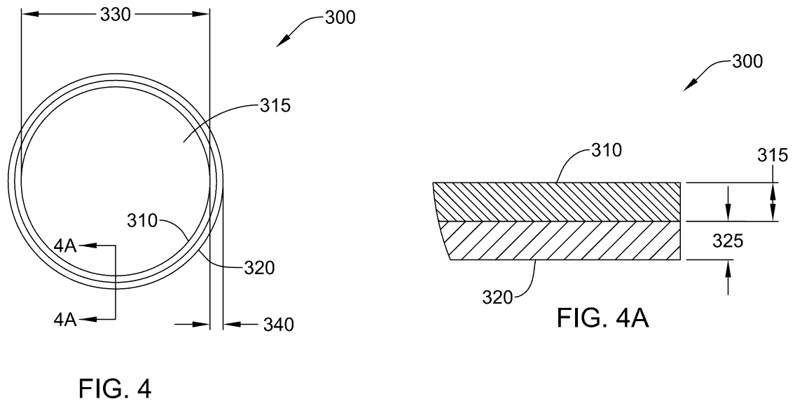

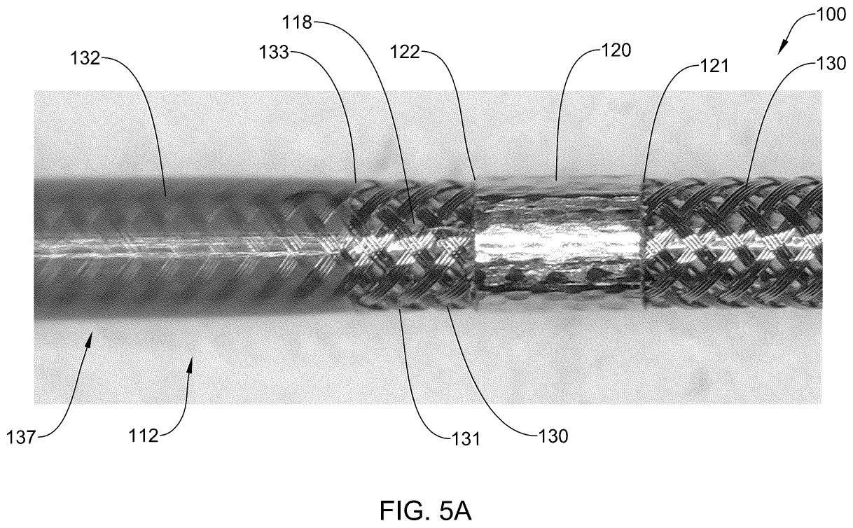

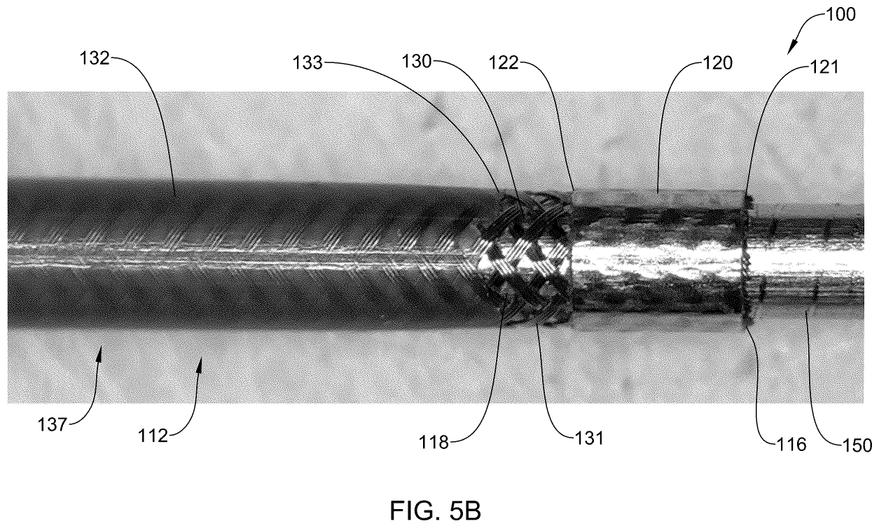

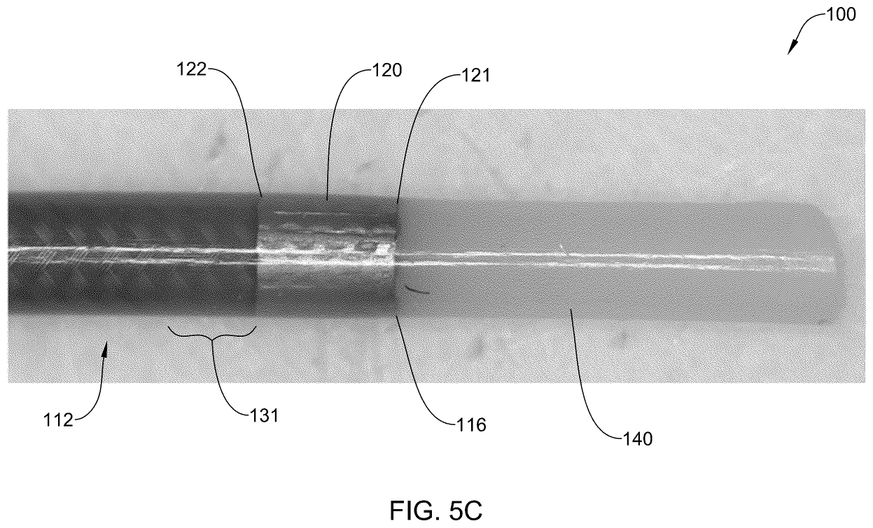

A distal tip 140 may be secured to the distal end 116 of the elongated tubular shaft 112 and extend distally therefrom. In some cases, the distal tip 140 may be a polymeric distal tip which may be formed from an elastomer (e.g., Pebax®), a thermoplastic polymer, polyether-block-amide-copolymer, or any other suitable polymer. In some cases, the distal tip 140 may be formed as a coextrusion that includes a first or inner layer formed of a first polymeric material and a second or outer layer formed of a different, second polymeric material, as discussed in reference to FIGS. 3 to 4A. In other instances, the distal tip 140 may be an extrusion of a single polymeric material. The distal tip 140 may be formed over the exposed region 131 of the middle layer 130 and the marker band 120, and extend distally beyond the distal end 116 of the elongated tubular shaft 112 to a distal end 141 of the distal tip 140.

For instance, the distal tip 140 may be extruded (including co-extrusion processes) over the exposed region 131 of the middle layer 130 and the marker band 120, with the extrusion extending distal of the distal end 116 of the elongated tubular shaft 112. Thus, the molten polymeric material of the distal tip 140 may flow into interstices 118 in the exposed region 131 of the middle layer 130 (e.g., flow into interstices 118 defined between braided filaments of the middle layer 130) during the extrusion process, and in some instances may be bonded to the inner layer 136 between a plurality of interstices 118 in the exposed region 131 of the middle layer 130, as referenced by 145 in FIG. 2B. In some cases, a proximal portion 142 of the distal tip 140 may radially surround and overlap with a distal portion 134, such as a distally tapered portion, of the outer layer 132. Thus, the proximal end 143 of the material forming the distal tip 140 (i.e., the proximal end 143 of the proximal portion 142 of the distal tip 140) may be located proximal of the distal end 133 of the outer layer 132.

FIG. 3 is a transverse cross-sectional view of an exemplary distal tip 200 of a catheter (e.g., catheter 10, 100), and FIG. 3A is an enlarged cross-section of the wall of the distal tip 200 of the catheter as in FIG. 3. The distal tip 200 may be considered to be an example of the distal tip 140. The distal tip 200 may include any desired inner diameter (ID) 230 sized to accommodate a 0.014-inch guidewire, a 0.018-inch guidewire, or a 0.035-inch guidewire, for example. For instance, the ID may be about 0.015 inches to about 0.017 inches, about 0.019 inches to about 0.022 inches, or about 0.034 inches to about 0.038 inches, for example. In some cases, the wall of the distal tip 200 may include a radial thickness 240 in a range of about 0.002 inches to about 0.003 inches, or any other suitable radial thickness.

In some instances, the distal tip 200 may be formed as a coextrusion that includes a first or inner layer 210 formed of a first polymeric material and a second or outer layer 220 formed of a different, second polymeric material, as shown in FIG. 3. In some cases, the distal tip 200 may be formed of a softer material (i.e., lower durometer) than other portions of the elongate shaft of the catheter (e.g., catheter 10), such as by using a polymer or elastomer having a shore hardness of less than 40D, for example. In some cases, the distal tip 200 may be formed from a polymer with a shore hardness of about 35D or less. In some instances, the inner layer 210 may be formed from a first polyether-block-amide-copolymer, and the outer layer 220 may be formed from a different, second polyether-block-amide-copolymer surrounding the first polyether-block-amide-copolymer. In some cases, the first polyether-block-amide-copolymer (e.g., inner layer 210) may be formed from a blend of a 63D Pebax® and bismuth subcarbonate (BiSubC), such as about 70% 63D Pebax® and about 30% bismuth subcarbonate. In some cases, the second polyether-block-amide-copolymer (e.g., outer layer 220) may be formed from a 25D Pebax® and barium sulfate (BaSO₄), such as about 80% 25D Pebax® and about 20% barium sulfate. These are just examples.

As shown in FIG. 3A, the inner layer 210 may include a radial thickness 215 in a range of about 0.0012 inches to about 0.0016 inches, or about 0.0014 inches, and the outer layer 220 may include a radial thickness 225 in a range of about 0.0008 inches to about 0.0012 inches, or about 0.0010 inches. In some cases, a ratio of the radial thickness 215 of the inner layer 210 to the radial thickness 225 of the outer layer 220 may be about 1.4:1. These are just examples.

FIG. 4 is a transverse cross-sectional view of an exemplary distal tip 300 of a catheter (e.g. catheter 10, 100), and FIG. 4A is an enlarged cross-section of the wall of the distal tip 300 of the catheter as in FIG. 4. The distal tip 300 may be considered to be an example of the distal tip 140. The distal tip 300 may include any desired inner diameter (ID) sized to accommodate a 0.014-inch guidewire, a 0.018-inch guidewire, or a 0.035-inch guidewire, for example. For instance, the ID may be about 0.015 inches to about 0.017 inches, about 0.019 inches to about 0.022 inches, or about 0.034 inches to about 0.038 inches, for example. In some cases, the wall of the distal tip 300 may include a radial thickness 340 in a range of about 0.002 inches to about 0.003 inches, or any other suitable radial thickness.

In some instances, the distal tip 300 may be formed as a coextrusion that includes a first or inner layer 310 formed of a first polymeric material and a second or outer layer 320 formed of a different, second polymeric material, as shown in FIG. 4. In some cases, the distal tip 300 may be formed of a softer material (i.e., lower durometer) than other portions of the elongate shaft of the catheter (e.g., catheter 10), such as by using a polymer or elastomer having a shore hardness of less than 40D, for example. In some cases, the distal tip 300 may be formed from a polymer with a shore hardness of about 35D or less. In some instances, the inner layer 310 may be formed from a first polyether-block-amide-copolymer, and the outer layer 320 may be formed from a different, second polyether-block-amide-copolymer surrounding the first polyether-block-amide-copolymer. In some cases, the first polyether-block-amide-copolymer (e.g., inner layer 310) may be formed from a blend of a 55D Pebax® and bismuth subcarbonate (BiSubC), such as about 70% 55D Pebax® and about 70% bismuth subcarbonate. In some cases, the second polyether-block-amide-copolymer (e.g., outer layer 320) may be formed from a 35D Pebax® and barium sulfate (BaSO₄), such as about 80% 35D Pebax® and about 20% bismuth subcarbonate. These are just examples.

As shown in FIG. 4A, the inner layer 310 may include a radial thickness 315 in a range of about 0.0010 inches to about 0.0014 inches, or about 0.0012 inches, and the outer layer 320 may include a radial thickness 325 in a range of about 0.0010 inches to about 0.0014 inches, or about 0.0012 inches. In some cases, a ratio of the radial thickness 315 of the inner layer 310 to the radial thickness 325 of the outer layer 320 may be about 1:1. These are just examples.

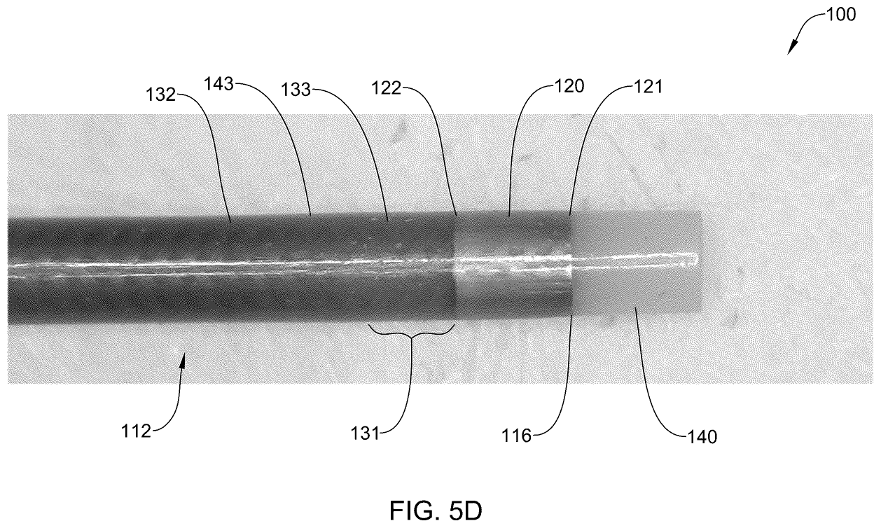

FIGS. 5A to 5D depict an illustrative method 400 of forming the catheter 100 having the distal tip 140. The method 400 may include disposing the braided middle layer 130 over the polymeric inner layer 136 (as shown in FIGS. 2 to 2B). For example, the braided middle layer 130 may be braided directly onto the inner layer 136 using a braiding apparatus. In other instances, the braided middle layer 130 may be braided separately and subsequently disposed around the inner layer 136. The marker band 120 may then be positioned over the braided middle layer 130 to secure the braided middle layer 130. For example, the marker band 120 may be swaged around the braided middle layer 130. The polymeric outer layer 132 may then be extruded over a proximal region 137 of the braided middle layer 130, with the inner layer 136 disposed within the middle layer 130, while leaving a distal portion 134 of the braided middle layer 130 proximal of the marker band 120 exposed from the outer layer 132, e.g., the exposed region 131, shown in FIG. 5A. In some instances, an outer diameter of a distal end region of the outer layer 132 may taper distally to the distal end 133 of the outer layer 132. In other words, a distal end region of the outer layer 132 may taper radially inward in a distal direction to the distal end 133 of the outer layer 132. The exposed region 131 of the braided middle layer 130 may extend between the distal end 133 of the outer layer 132 and the proximal end 122 of the marker band 120. Thus, the middle layer 130 may extend distally beyond the distal end 133 of the outer layer such that the outer layer 132 does not surround or extend over the exposed region 131 of the middle layer 130.

As shown in FIG. 5B, portions of the braided middle layer 130 and the inner layer 136 that extend distal of a distal end 121 of the marker band 120 may be cut away or otherwise removed, such that a distalmost end of the braided middle layer 130 and the inner layer 136 lie substantially flush with the distal end 121 of the marker band 120. In other words, the distal end 116 of the elongated tubular shaft 110 may lie substantially flush with the distal end 121 of the marker band 120. As shown in FIG. 5B, a mandrel 150 may be inserted into the lumen 115 at the distal end 116 of the elongated tubular shaft 112, and the mandrel 150 may extend distal of the distal end 116 of the elongated tubular shaft 112.

As shown in FIG. 5C, the distal tip 140 may be extruded (including co-extrusion of multiple layers of the distal tip 140) over the mandrel 150, as well as over the exposed region 131 of the braided middle layer 130 and the marker band 120. The distal tip 140 may be extruded over the exposed region 131 of the braided middle layer 130 and the marker band 120, and may extend distally beyond the distal end 121 of the marker band 120, as shown in FIG. 5C. For instance, the distal tip 140 may be extruded (including co-extrusion processes) over the exposed region 131 of the middle layer 130 and the marker band 120, with the extrusion extending distal of the distal end 116 of the elongated tubular shaft 112. Thus, the molten polymeric material of the distal tip 140 may flow into interstices 118 in the exposed region 131 of the middle layer 130 (e.g., flow into interstices 118 defined between braided filaments of the middle layer 130) during the extrusion process, and in some instances may be bonded to the inner layer 136 between a plurality of interstices 118 in the exposed region 131 of the middle layer 130. In some cases, a proximal portion 142 of the distal tip 140, such as a proximally tapered portion of the distal tip 140, may radially surround and overlap with a distal portion 134, such as a distally tapered portion, of the outer layer 132. The formed lap joint may thereby provide an increased tip tensile strength at the interface between the distal tip 140 and the elongated tubular shaft 112.

A distal region of the distal tip 140 may taper to a smaller outer diameter as the distal tip 140 extends distal of the distal end 121 of the marker band 120, and thus as the distal tip 140 extends distal of the distal end 116 of the elongate tubular shaft 112.

As shown in FIG. 5D, the mandrel 150 may be removed and the distal tip 140 may then be cut to a desired length, such as a length of about 1 millimeter (mm). In some cases, the distal tip 140 may be cut to have a length of about 2.0 mm, about 1.7 mm, about 1.5 mm, about 1.3 mm, about 1.0 mm, or any other suitable length.

In some cases, as discussed with reference to FIGS. 3 to 4A, the distal tip 140 may be formed as a coextrusion that includes a first or inner layer and a second or outer layer. In some cases, the distal tip 140 may be formed of a softer material (i.e., lower durometer) than other portions of the elongate shaft of the catheter (e.g., catheter 10), such as by using a polymer or elastomer having a shore hardness of less than 40D, for example. In some cases, the distal tip 140 may be formed from a polymer with a shore hardness of about 35D or less. In some instances, the inner layer may be formed from a first polyether-block-amide-copolymer, and the outer layer may be formed from a different, second polyether-block-amide-copolymer radially surrounding the first polyether-block-amide-copolymer.

The above described method 400 of forming the catheter 100 having the distal tip 140 may be considered to be an example of a lap joint bonding process. The method 400 may serve to provide a catheter 100 having a distal tip (e.g., distal tip 140) with a reduced entry profile, improve tip tensile strength, and increase the extension at break. For example, the process of bonding the distal tip 140 to the catheter 100 includes extruding the distal tip 140 over the distal end 116 of the elongated tubular shaft 112 and the marker band. The material forming the distal tip 140 may extend into the plurality of interstices 118 in the exposed region 131 of the middle layer 130 (e.g., within interstices 118 of the braided structure and, in some instances, contact the inner layer 136, thereby forming a bond between the distal tip 140 and the inner layer 136. This provides a strong bond between the distal tip 140 and the distal end 116 of the elongated tubular shaft 112, which is contrary to traditional methods of coupling a distal tip to an elongated shaft of a catheter, such as a butt joint bond.

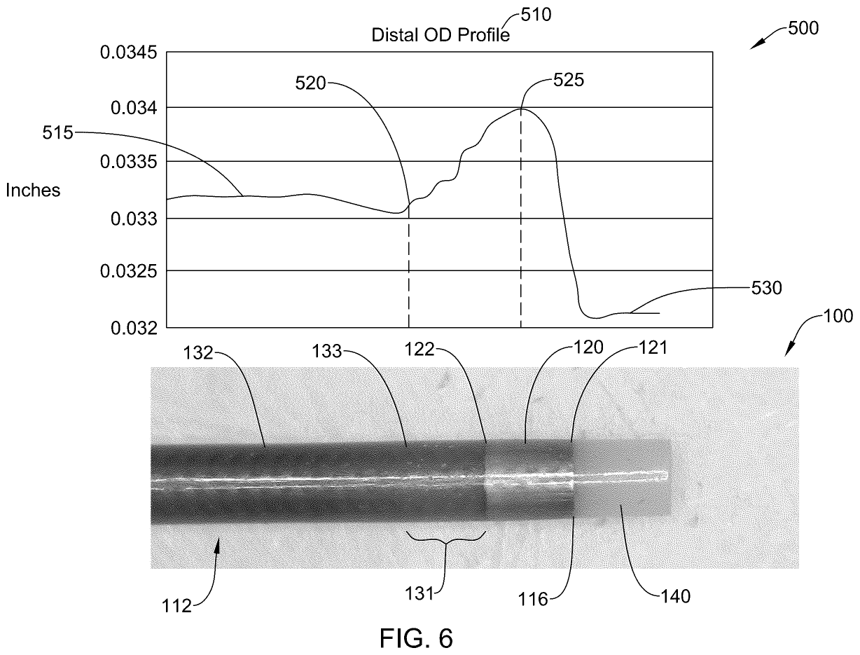

FIG. 6 is an illustrative graph 500 depicting a distal outer diameter (OD) profile 510 of a catheter, e.g., catheter 100. As previously discussed, the OD of the distal end 133 of the outer layer 132 tapers distally as the outer layer 132 terminates proximal to the marker band 120. This is illustrated in FIG. 6 at 515, where the elongated tubular shaft 112 includes an OD of about 0.0332 inches, for example, and at 520, which is just proximal of the exposed region 131, where the elongated tubular shaft 112 includes an OD of about 0.0330 inches, for example. In some cases, the OD of the elongated tubular shaft 112 may increase in the area including the marker band 120, as the distal tip 140 has been formed over the exposed region 131 and the marker band 120, thereby increasing the OD of the elongated tubular shaft 112 surrounding the marker band 120 to about 0.0340 inches, for example, as indicated at 525 on the graph 500. The distal tip 140 may taper distally beyond the marker band 120 such that the distal tip 140 may include a smaller OD than the elongated tubular shaft 112. For example, the distal tip 140 may include an OD of about 0.0321 inches, as referenced at 530. While the graph 500 illustrates the OD profile of the elongated tubular shaft 112 and the distal tip 140 ranging between about 0.032 inches to about 0.034 inches, it may be contemplated that the OD profile of the elongated tubular shaft 112 and the distal tip 140 may include any other suitable range. For example, the OD profile may be in a range of about 0.03 inches to about 0.04 inches. These are just examples.

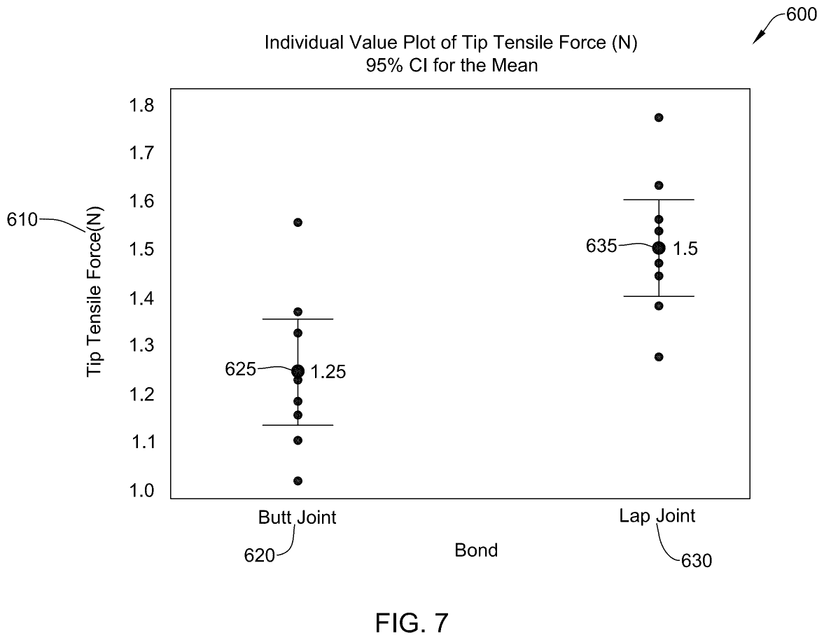

FIG. 7 is an illustrative graph 600 comparing a tensile force 610 of a catheter with a butt joint 620 versus a lap joint 630 formed in accordance with this disclosure. As discussed above with reference to FIG. 5, the method (e.g., method 400) of forming the distal tip 140 via the lap joint 630 may improve the tip tensile strength of the distal tip 140. For example, as shown in the graph 600, when a butt joint 620 is used to couple a distal tip to a catheter, the maximum tip tensile force 610 the butt joint 620 can withstand falls within a range of about 1.02 N (Newtons) to about 1.6 N, with an average value 625 of about 1.25 N, for example. However, when the lap joint 630 as described herein is used to secure the distal tip 140 to the catheter 100, the maximum tip tensile force 610 the lap joint 630 can withstand falls within a range of about 1.3 N to about 1.78 N, with an average value 635 of about 1.5 N, for example. Thus, the lap joint formed in accordance with this disclosure may have a tensile strength of 1.3 N or greater, 1.5 N or grater, or 1.7 N or greater.

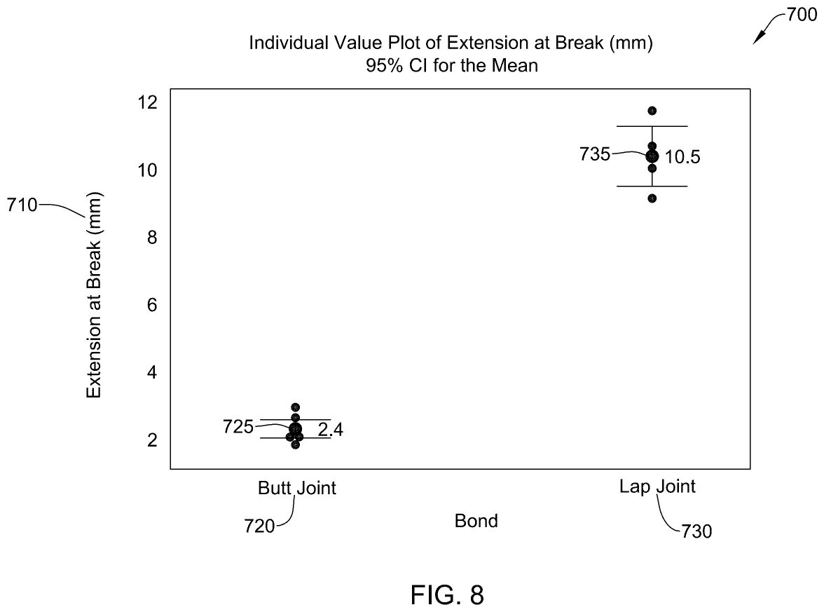

FIG. 8 is an illustrative graph 700 comparing an extension at break 710 for a catheter with a butt joint 720 versus a lap joint 730 formed in accordance with this disclosure. As discussed above with reference to FIG. 5, the method (e.g., method 400) of forming the distal tip 140 via the lap joint 730 may increase the extension at break 710 for the distal tip 140. For example, as shown in the graph 700, when a butt joint 720 is used to couple a distal tip to a catheter, the maximum elongation at break for the distal tip falls within a range of about 1.8 mm (millimeters) to about 3 mm, with an average value 725 of about 2.4 mm, for example. However, when the lap joint 730 as described herein is used to secure the distal tip 140 to the catheter 100, the maximum elongation at break of the distal tip 140 falls within a range of about 9 mm to about 11.7 mm, with an average value 735 of about 10.5 mm, for example. Thus, the lap joint formed in accordance with this disclosure may have an elongation at break of 9 mm or greater, 10 mm or grater, or 11 mm or greater.

The catheter 10, 100 and various components thereof, may be manufactured according to essentially any suitable manufacturing technique including extruding, co-extruding, molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304L, and 316LV stainless steel; mild steel; nickel alloys such as linear-elastic and/or super-elastic nitinol; nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A catheter comprising:
an elongated tubular shaft having a distal end, a proximal end, and a lumen extending therethrough, the elongated tubular shaft including:
a polymeric outer layer;
a polymeric inner layer; and
a braided middle layer positioned between the inner layer and the outer layer;
a marker band positioned adjacent the distal end of the elongated tubular shaft and surrounding the middle layer; and
a polymeric distal tip attached to the distal end of the elongated tubular shaft;
wherein a distal end of the outer layer terminates proximal to the marker band such that the middle layer includes an exposed region that is exposed from the outer layer between the distal end of the outer layer and a proximal end of the marker band;
wherein the distal tip is formed over the exposed region of the middle layer and the marker band, and extends distally beyond the distal end of the elongated tubular shaft.

2. The catheter of claim 1, wherein the outer layer terminates at a distance in the range of about 0.5 mm (millimeters) to about 1.5 mm proximal of the marker band.

3. The catheter of claim 1, wherein the distal tip provides a tensile strength in the range of 1.42 N (Newtons) to 1.55 N.

4. The catheter of claim 1, wherein the distal tip provides a tensile strength of about 1.5 N.

5. The catheter of claim 1, wherein the distal tip is formed from a polyether-block-amide copolymer.

6. The catheter of claim 1, wherein a proximal portion of the distal tip surrounds and overlaps with a distal portion of the outer layer.

7. The catheter of claim 1, wherein the distal tip is formed from a coextrusion comprising a first polyether-block-amide copolymer layer and a second polyether-block-amide copolymer layer surrounding the first polyether-block-amide copolymer layer.

8. The catheter of claim 7, wherein a ratio of a radial thickness of the first polyether-block-amide copolymer layer to the second polyether-block-amide copolymer layer is about 1:1.

9. The catheter of claim 7, wherein a ratio of a radial thickness of the first polyether-block-amide copolymer layer to the second polyether-block-amide copolymer layer is about 1:1.4.

10. A method of forming a catheter having a distal tip, the method comprising:
disposing a braided middle layer over a polymeric inner layer;
positioning a marker band over the braided middle layer;
extruding a polymeric outer layer over a proximal region of the braided middle layer while leaving a distal region of the braided middle layer exposed from the outer layer, the exposed distal region of the braided middle layer extending between a distal end of the outer layer and a proximal end of the marker band;
cutting away portions of the braided middle layer and the inner layer extending distal of a distal end of the marker band;
thereafter, extruding a distal tip over the exposed distal region of the braided middle layer and the marker band;
wherein the distal tip extends distally beyond the distal end of the marker band.

11. The method of claim 10, wherein the distal tip is formed from a coextrusion comprising a first polyether-block-amide copolymer layer and a second polyether-block-amide copolymer layer surrounding the first polyether-block-amide copolymer layer.

12. The method of claim 10, wherein the distal tip is bonded to the inner layer between a plurality of interstices in the exposed distal region of the middle layer.

13. The method of claim 10, wherein the distal tip provides a tensile strength in the range of 1.42 N (Newtons) to 1.55 N.

14. The method of claim 10, wherein the outer layer terminates at a distance in the range of about 0.5 mm (millimeters) to about 1.5 mm proximal of the marker band.

15. The method of claim 10, wherein an outer diameter of a distal end region of the outer layer tapers distally.

16. The method of claim 10, wherein a proximal portion of the distal tip surrounds and overlaps with the distal end region of the outer layer.

17. A catheter comprising:
an elongated tubular shaft having a distal end, a proximal end, and a lumen extending therethrough, the elongated tubular shaft including:
a polymeric inner layer having an inner surface defining the lumen of the elongated tubular shaft, the inner layer extending continuously from the proximal end of the elongated tubular shaft to the distal end of the tubular shaft;
a braided middle layer surrounding the inner layer, the middle layer extending continuously from the proximal end of the elongated tubular shaft to the distal end of the elongated tubular shaft;
a polymeric outer layer surrounding the middle layer, the outer layer extending continuously from the proximal end of the elongated tubular shaft to a distal end of the outer layer that is located proximal of the distal end of the elongated tubular shaft such that the middle layer includes a distal end region extending distal of the distal end of the outer layer; and
a polymeric distal tip extruded over the distal end region of the middle layer and extending distally beyond the distal end of the elongated tubular shaft.

18. The catheter of claim 17, wherein a proximal portion of the distal tip surrounds and overlaps with a distal portion of the outer layer.

19. The catheter of claim 18, wherein an outer diameter of the distal portion of the outer layer tapers distally.

20. The catheter of claim 18, further comprising a marker band positioned adjacent the distal end of the elongated tubular shaft and surrounding the middle layer, wherein the distal tip extends over and surrounds the marker band.
